(19) United States
(12) Patent Application Publication
(10) Pub. No.: US 2005/0042241 A1
(43) Pub. Date: Feb. 24, 2005

(54) Dr. Bob's Arthritis Cream

(76) Inventors: Robert Cusic, Dickinson, ND (US); Steve Irsfeld, Dickinson, ND (US)

Correspondence Address: Robert Cusic;Steve Irsfeld, 629 Tenth Avenue West Dickinson, ND 58601, 33 West Ninth Street Dickinson, ND 58601, Dickinson, ND, 58601, US

(21) Appl. No.: 10/604,849

(22) Filed: Aug. 21, 2003

(51) Int. Cl. A61K 31/137

(52) U.S. Cl. 424/401; 514/649

(57) ABSTRACT

The invention provides a topical cream to treat the pain and inflammation found in a variety of medical disorders. These disorders include, but are not limited to, medical conditions such as arthritis, bursitis and tendonitis. The invention provides two medications, DMSO and PLO gel, to bring pain and inflammation reducing drugs through intact skin. The pain and inflammation reducing drugs are diphenhydramine and ketoprofen (or other non-specific cox inhibitor). It is further anticipated that this compounded formula could be sold O.T.C. (over the counter, without a prescription needed).

### DETAILED DESCRIPTION

Detailed Description/Ingredients: The formula for 100 grams cream is as follows:

Ketoprofen USP 10 gm powder (or other similar nonspecific cox inhibitor); DMSO 10 gm; Diphenhydramine HCL 2 gm; Lecithin Isopropyl Palmitate solution 22 ml/100 gm; Ethoxy Diglycol 5 ml/100 gm; QS with Pluronic 20% gel to 100 gm. The pluronic 20% gel is made from 20 gm of Poloxamer 407NF, 0.3 gm Potassium sorbate NF, QS to 100 ml with purified water USP. The Lecithin/Isopropyl Palmitate Solution 220 ml consists of Lecithin Soya granular 100 gm and Isopropyl Palmitate NF 100 gm (117 ml). Sorbic acid NF-FCC powder 0.66 gm.

1. A topical cream applied to the skin which relieves the pain of many types of arthritis, tendonitis, and bursitis. The cream is compounded to allow delivery of a pain and anti-inflammatory combination, through the skin, directly to inflamed joints, bursae, and tendons. Ketoprofen is the anti-inflammatory used, but other non-specific cox inhibitors may also be effective. Ketoprofen has both anti-inflammatory and pain relieving effect. For additional pain relief, diphenhydramine has been added. Diphenhydramine has long been recognized as an effective local anesthetic in the presence of an allergy to both “esther” and “amide” classes of local anesthetics. The “delivery vehicle” for these pain relieving drugs is dimethylsulfoxide (DMSO) and what is commonly called “PLO gel” (a mixture of pluronic gel and lecithin). DMSO has long been known to penetrate intact skin. It may also have some direct, but brief, pain relief. More recently, PLO gel has been used successfully to bring medications, such as antiemetics, through the skin. This combination allows the longer acting pain relievers ketoprofen and diphenhydramine to reach underlying inflamed tissues. The cream is especially effective when combined with an oral anti-inflammatory. It may be applied up to t.i.d. and should be rubbed in well. It may prove (not yet tested) to be safe in patients on an anti-coaggulant such as coumadin (for whom systemic anti-inflammatories may be contraindicated). It has so far proven to provide consistent pain relief to patients with osteoarthritis of the knee and lateral humeral epicondylitis (“tennis elbow”). Improvement to other inflammatory conditions has been found but with less consistency. It is not to be used in inflammatory conditions caused by infection and should be avoided in patients with allergies to “Sulfas” or aspirin related products. Development of skin irritation or a rash should cause discontinuation of the product.
